(19) United States
(12) Patent Application Publication
(10) Pub. No.: US 2018/0116832 A1
(43) Pub. Date: May 3, 2018

(54) STENT AND STENT SET

(71) Applicant: Jayandiran Pillai, Johannesburg (ZA)

(72) Inventor: Jayandiran Pillai, Johannesburg (ZA)

(21) Appl. No.: 15/572,771

(22) PCT Filed: Apr. 29, 2016
(86) PCT No.: PCT/IB2016/052433

§ 371 Date: Nov. 8, 2017

(30) Foreign Application Priority Data
May 8, 2015 (ZA) 2015/02864

(51) Int. Cl. A61F 2/856; A61F 2/852; A61F 2/07

(52) U.S. Cl. CPC A61F 2/856; A61F 2/852; A61F 2/07; A61F 2250/0096; A61F 2250/0029; A61F 2250/006; A61F 2250/0018

(57) ABSTRACT

This invention relates to a stent and a stent set. In accordance with this invention there is provided a stent comprising a tubular stent body made of mesh and an opening in the tubular body.

### CROSS REFERENCE TO RELATED APPLICATIONS

This application is a U.S. National Stage Application under 35 U.S.C. 371 of International Application No. PCT/IB2016/0542433 filed Apr. 29, 2016, which application claims priority to South Africa Application No. 2015/02864, filed May 8, 2015, the contents of which are hereby incorporated by reference in their entirety into the present disclosure.

### FIELD OF THE INVENTION

This invention relates to a stent and a stent set.

### BACKGROUND TO THE INVENTION

In medicine, a stent is a tube or other device placed in the body to create a passage between two hollow spaces or to keep open such a passage. There are a wide variety of stents used for different purposes, from expandable coronary, vascular and biliary stents, to simple plastic stents used allow the flow of urine between kidney and bladder. Stent is also used as a verb to describe the placement of such a stent, particularly when a disease such as atherosclerosis has pathologically narrowed a body structure such as an artery. Coronary stents are placed during a percutaneous coronary intervention, also known as angioplasty. The most common use for coronary stents is in the coronary arteries, into which a bare-metal stent, a drug-eluting stent, a bio-absorbable stent, a dual-therapy stent (combination of both drug and bioengineered stent), or occasionally a covered stent is inserted.

Vascular stents are commonly placed as part of peripheral artery angioplasty. Common sites treated with peripheral artery stents include the carotid, iliac, and femoral arteries. Because of the external compression and mechanical forces subjected to these locations, flexible stent materials such as nitinol are used in a majority of peripheral stent placements.

A stent graft or covered stent is type of vascular stent with a fabric coating that creates a contained tube but is expandable like a bare metal stent. Covered stents are used in endovascular surgical procedures such as endovascular aneurysm repair. Stent grafts are also used to treat stenosis in vascular grafts and fistulas used for haemodialysis.

Various patents exist and various patent applications have been filed for stents.

International patent application number PCT/EP2013/073484 published under publication number WO 2014072501 A3 in the name of Georg-August-Universität Göttingen Stiftung Öffentlichen Rechts Universitätsmedizin, entitled “Endovascular stent grafts, stent, stent-in-stent system, and kit” discloses, in a first aspect, an endovascular stent graft, which comprises a one-piece stent. In a second aspect, the invention relates to an additional endovascular stent graft. In an additional aspect, the invention relates to a stent-in-stent system comprising the aforementioned endovascular stent grafts. In a fourth aspect, the invention relates to the use of one or both endovascular stent grafts and/or the stent-in-stent system for the treatment of aneurysms and/or in the event of vascular leakages. In another aspect, the invention relates to an endovascular stent that has at least one longitudinal end that is provided with fixing elements that are arranged peripherally at the longitudinal end. In a last aspect, the invention relates to a kit, comprising one of the two or both endovascular stent grafts according to the invention and the endovascular catheter according to the invention. The applicant discloses the use of two stent bodies.

International patent application number PCT/US2004/028530 publication number WO 2005023149 A3 in the name of Bolton Medical Inc., entitled “Stent graft, stent graft delivery system and kit and method for implanting the stent graft” discloses a vascular repair device including a tubular graft body and a structural framework having at least two stents connected to the graft body. Stents of the structural framework can each be respectively connected to graft body adjacent the proximal and ends and the support member is shorter than the separation distance there between to form a gimbal at least at one end. A first stent is connected along an entirety thereof and a second stent is connected at distal apices thereof. Distal apices of the second stent have radii of curvature smaller than proximal apices. A curved longitudinal support member can be connected to the graft body, the member being substantially symmetrical with respect to the centreline. The support member can be connected to the graft body independent of the structural framework. At least one of the ends of the support member can have a curved longitudinal extremity.

US patent application number 20100016947 A1 in the name of John Daniel Dobak III, entitled “Systems and methods for creating customized endovascular stents and stent grafts” discloses a system and method for making a customized stent or stent graft, including the steps of obtaining a digital image of the endoluminal shape of an artery or the blood flow channel of an aneurysm, processing the obtained image to create a three dimensional model of the shape or channel, and fabricating a scaffold around the model such that the scaffold substantially conforms to the model. Dobak states that one problem with current endovascular grafts and stents is a lack of conformation with the lumen of the vessel into which they are placed. Vessels may be curved, or tortuous, bifurcated, and can have changing diameter. Current stents and grafts differ in their flexibility and ability to conform to the vessel anatomy. This can lead to several problems.

Whilst Dobak proposes an elegant and/or advanced solution to achieve lumen conformity, the solution may not always be readily available or affordable.

International patent application number WO 2000062708 A1 entitled “A multi-section stent” in the name of the Mayo Foundation for Medical Education and Research discloses a multi-section stent which includes a connecting structure that allows the stent sections to move and flex relative to one another. For deployment and positioning, the connecting structure connects the multiple stent sections and holds the stent sections substantially stationary relative to one another. Following deployment, the connecting structure allows the multiple stent sections to move relative to one another. Movable stent sections enable flexure of the stent upon deployment within a body lumen. This flexing structure allows better conformance of the stent to the shape of the body lumen, and exerts less overall pressure against the lumen wall, reducing the potential for trauma. Upon deployment, the multiple stent sections may be completely detached from one another. Alternatively, the stent sections may remain partially connected in a manner that allows substantial independent movement. The connecting structure can be manufactured to separate upon deployment, for example, by breaking or degrading within the body lumen in which the stent is positioned.

US patent application number 20100262216 A1 entitled “Stent having a C-shaped body section for use in a bifurcation” in the name of Medtronic Vascular, Inc. discloses a bifurcation stent system includes a pair of self-expanding stents. Each stent has a C-shaped body section having a generally semi-circular cross-section along its length and an O-shaped body section having a circular cross-section along its length. The stents are deployed in vivo such that the edges of the C-shaped body sections abut each other to form a tubular scaffold in a Y-shaped formation that conforms to the bifurcation. In order to connect the stents in vivo, the C-shaped body sections are configured to include a ball and socket connection there between. The C-shaped body sections align and abut to form a tubular scaffold that extends in the main vessel of the bifurcation, while the O-shaped body sections are tubular scaffolds that extend into the respective branch legs of the bifurcation. The tubular scaffold formed by the two cooperative C-shaped sections do not always fit snugly in the human body and, as a result, resist blood flow leading to a host of disadvantages.

### OBJECT OF THE INVENTION

It is an object of this invention to provide a stent and a stent set of the type described above.

### SUMMARY OF THE INVENTION

In accordance with this invention there is provided a stent comprising a tubular stent body made of mesh and an opening in the tubular body.

There is provided for the mesh to be made of metal. The tubular body has opposed open ends.

The opening is formed by a discontinuation of the mesh body. Alternatively, the opening is formed by a cutaway section of the mesh body. Further alternatively, the mesh has larger openings in non-disease areas thereof.

The non-disease areas may be an end region or both end regions, a central annular region and/or a side region or combination of these.

Stent bodies having uniform mesh openings may be provided. Different stents with different sized but uniform stent openings may be provided. These stent may still include one openings for receiving part or an end of another stent therein.

There is provided for the opening to be larger than mesh openings as defined by the metallic mesh.

There is provided for the stent to have a marker.

There is further provided for the marker to be located on a free end of a strand which extends from the metallic mesh.

This invention extends to a stent set comprising a stent having a tubular stent body made of the metallic mesh with an opening in the body and a second stent; and end of the second stent being receivable into an end of the stent.

The set may include a third stent.

There is provided for the second and third state to be made of the metallic mesh.

A yet further feature of the invention provides for anyone of the opposing ends of the second or third stents are receivable in any one of the open ends of the tubular stent or in the opening in the body of the stent.

These and other features of invention are described in more detail below.

### BRIEF DESCRIPTION OF THE DRAWINGS

One embodiment of the invention is described below by way of example only and with reference to the drawings in which:

FIG. 1 shows a front view of a stent set;

FIG. 2 shows a front view of a stent of the stent set;

FIG. 3 shows a side view of the stent of FIG. 2 in an unexpanded condition;

FIG. 4 shows a rear view of the stent of FIGS. 2 and 3 in an unexpanded condition;

FIG. 5 shows a perspective view of part of the stent set inserted in an artery;

FIG. 6 shows a perspective view of 3 parts of the stent set inserted in an artery;

FIG. 7 shows a second embodiment of a stent;

FIG. 8 shows a third embodiment of a stent;

FIG. 9 shows a fourth embodiment of a stent;

FIG. 10 shows the stent of FIG. 7 in use; and

FIG. 11 shows the stent of FIG. 7 in use, in a reversed orientation; and

FIG. 12 shows alternate stents in use.

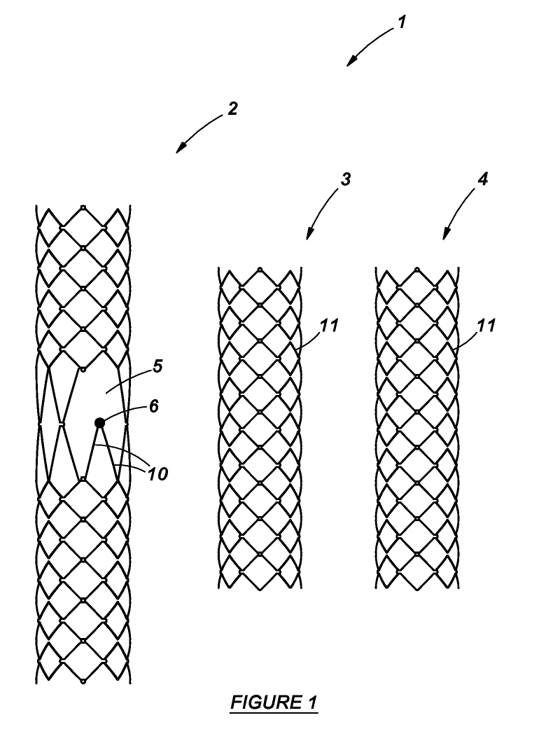

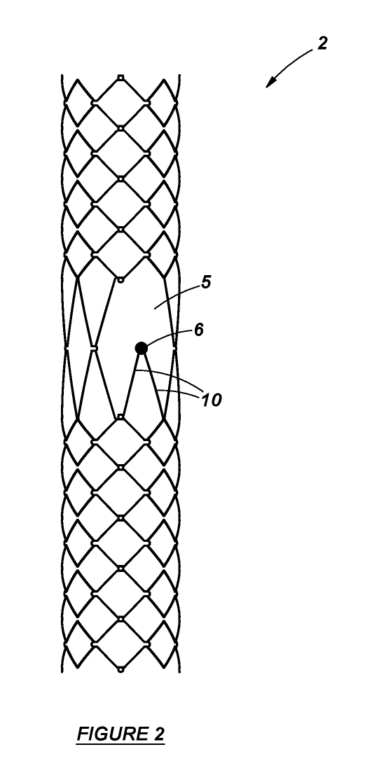

### DETAILED DESCRIPTION OF THE DRAWINGS

With reference to the drawings, in which like features are indicated by like numerals, a stent set is generally indicated by reference numeral 1.

The stent set 1 consists of a stent 2, a second stent 3 and a third stent 4. Each of these stents are made of a metallic mesh 11 and are substantially tubular with open ends.

Stent 2 has an opening 5 therein defined by a discontinuation of the mesh. The opening 5 is larger than any of the mesh openings defined by the mesh 11.

Stent 2 includes an x-ray marker 6. In this embodiment, the x-ray marker 6 is located where two free ends of two strands 10 meet. The two strands are elongate extensions from the mesh body.

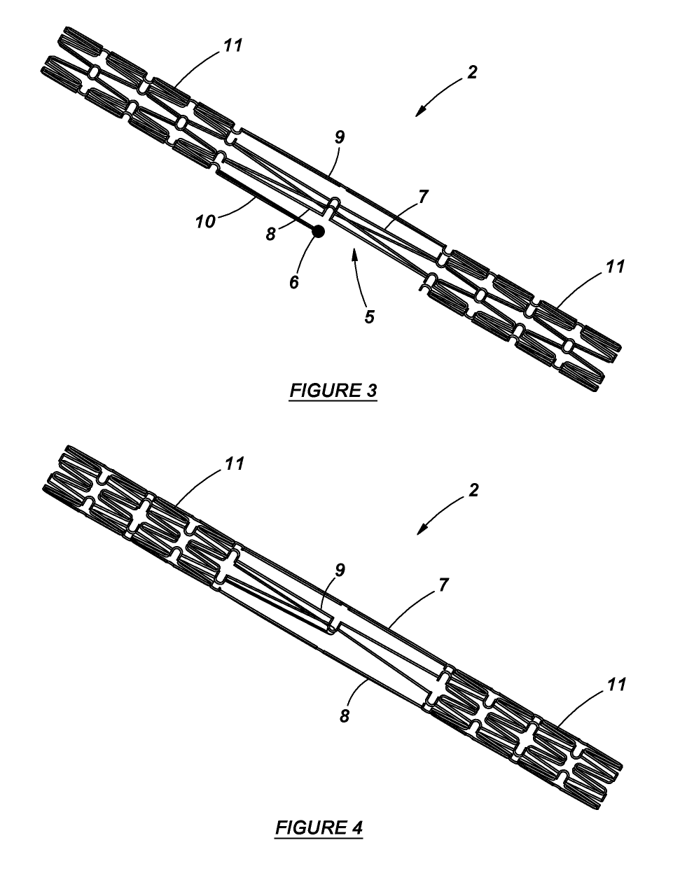

FIGS. 3 and 4 show the stent 2 in its pre-expanded condition. The stent is expanded with a balloon device after the insertion into the body of a patient as is known in the art.

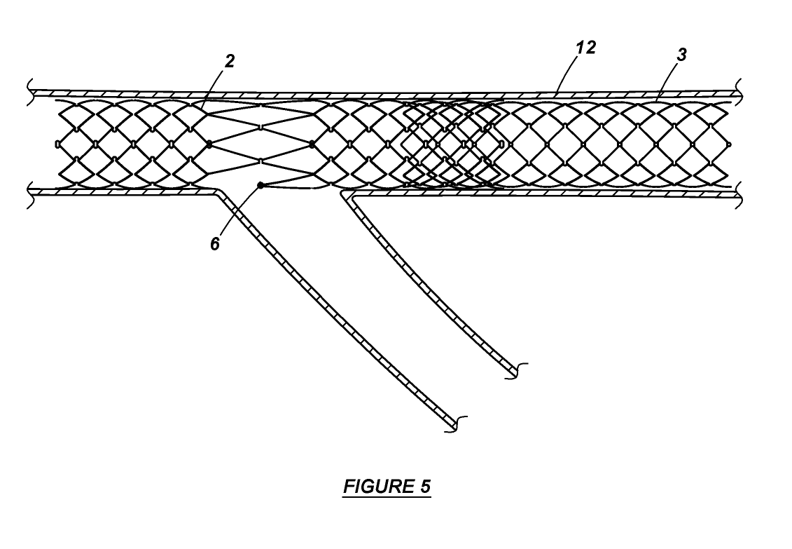

FIG. 5 shows the stent and in the second stent inserted in an artery (vessel) 12 as is known in the art. One end of the second stent is inserted in a free end of the stent 2.

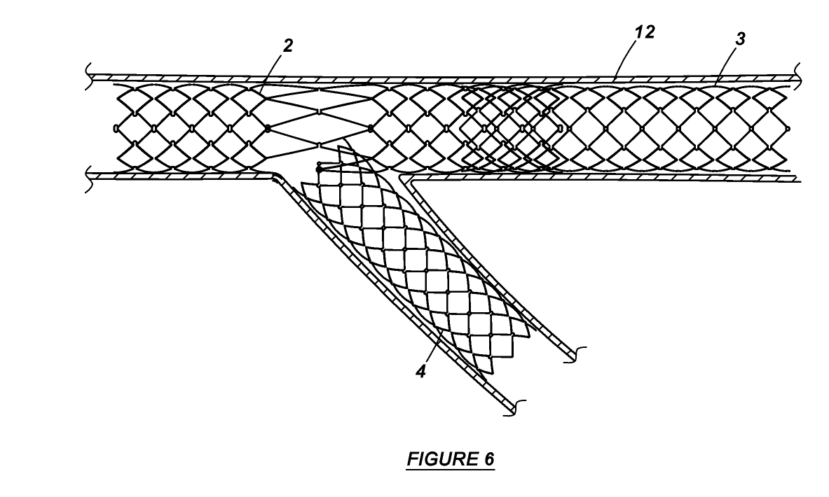

FIG. 6 shows a similar situation as that of FIG. 5. However, one end of the third stent 4 is inserted in the opening 5 of the stent 2. In this manner, stents 2 and 4 for a Y-shaped composite stent in a bifurcation. The opening of stent 2 locates in the opening of the bifurcation and faces towards the one vessel that joins in that bifurcation and in which vessel the stent 2 is not located.

In use, the stent 2 is implanted in a patient as is known in the art. Different parts of the stent may be inflated two different sizes as may be required, as is also known in the art. The stent is inserted using the marker 6 to ensure that it is properly in position with the opening 5 facing towards a first bifurcation so that the one end of the stent 2 locating the main artery and its opposite, second end, locates in the second bifurcation. With the opening 5 facing the first split artery (bifurcation) unrestricted flow of blood is possible into that split artery from the first end of the stent 2. The second end of the stent extends into the second bifurcation, which would be the more damaged or diseased side of the artery or bifurcation. If the stent 2 does not extend far enough into the second bifurcation, the surgeon may elect to extend the stent by inserting one end of the second stent 3 into the end of the stent that locates in the second bifurcation.

If a stent needs to be placed in the first bifurcation, and end of the third stent 4 is placed in the opening 5 of the stent 2.

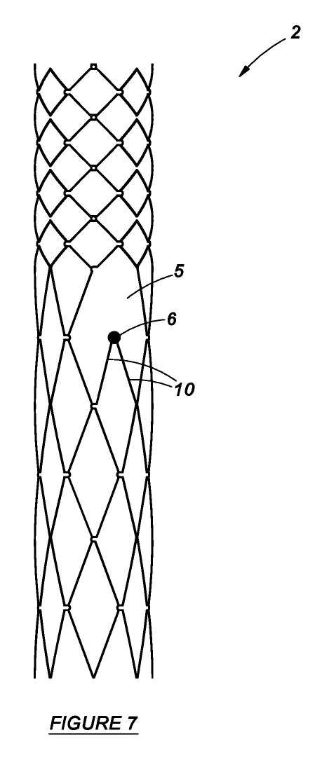

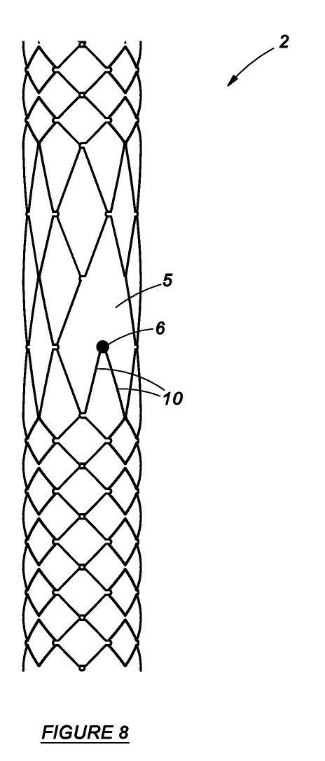

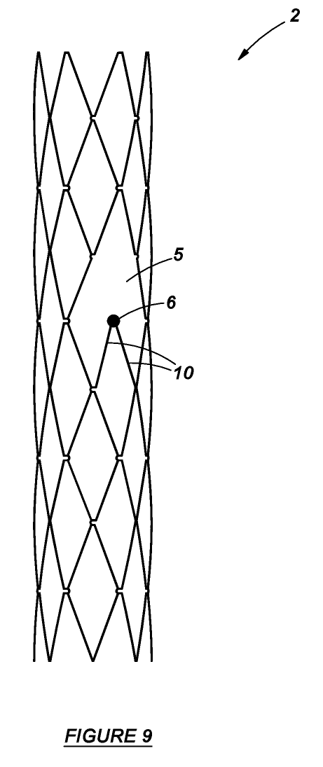

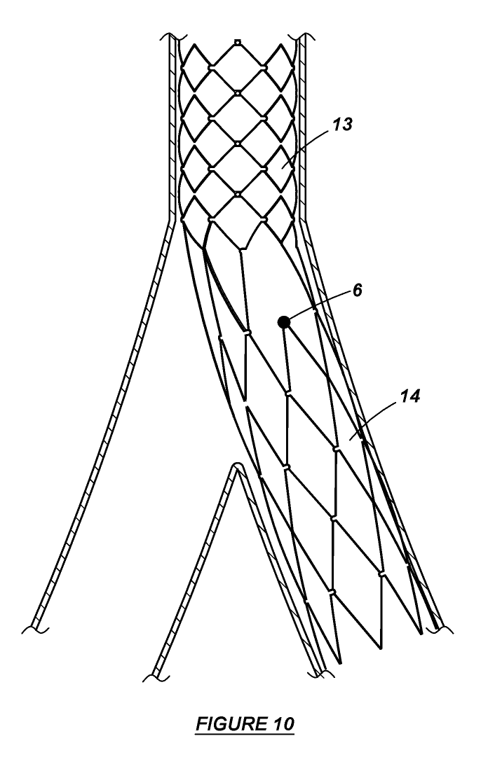

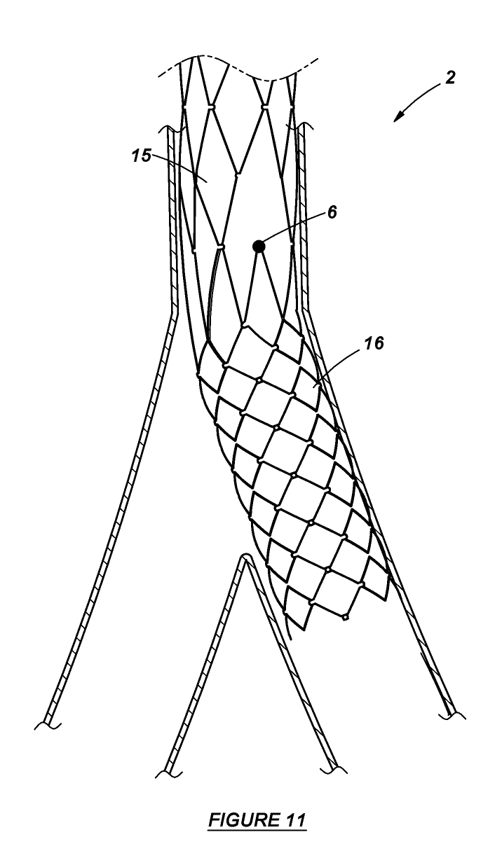

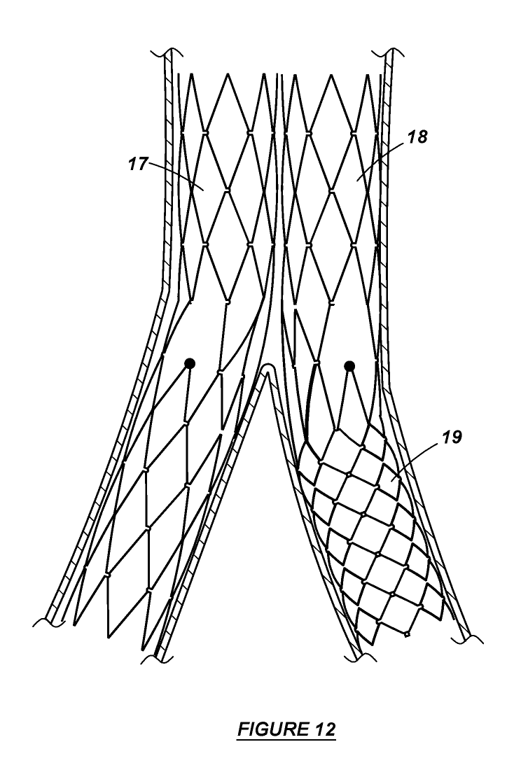

With reference to the stents of FIGS. 7 to 12, stents having different mesh size openings can be used. These stents may be made of a mesh body having varying size mesh openings. End regions may have larger openings that middle sections. Alternatively, side sections or centre sections may have different size openings than the rest of the stent body. The choice of the stent used will be guided by the area or size of the disease or damage of a vessel. Smaller stent openings (for example at 13, 16 and 19) will be used to cover diseased areas whilst the larger openings (for example 14, 15, 17 and 18) locate in healthy areas. The use of two stents as shown in FIG. 12 where the upper section 18 of one stent has relatively large openings (and smaller openings at it lower end 19) and the other stent 17 has the same openings over the whole stent body, turbulence will be less than with smaller openings in this upper area.

It envisaged that the stent and stent set described herein will be convenient to use as it will provide a surgeon with various options. The stent set, wind ends thereof are inserted into each other and into the opening as described above, will suffice to support the main artery and the arteries that split therefrom [“the bifurcations” or “the bifurcation arteries”]. If the surgeon finds that is not necessary or advisable to use the complete set consisting of the 3 stents, he or she may choose to use only one of the 3 stents. It is envisaged that stent 2 will mostly be used together with a stent extending further from an end of stent 2 and/or out of the opening 5 of stent 2.

The opening 5 can be a number of openings (one or more) in the mesh body which opening/s are enlarged mesh opening/s i.e. opening/s in the mesh that are larger than the rest of the mesh openings.

The invention is not limited to the precise details as described herein. It will be appreciated by those skilled in the art that many embodiments are possible without departing from the scope of the invention.

1. A stent comprising a tubular stent body having opposite open ends, made of a mesh and having an opening in the tubular body.

2. The stent of claim 1, wherein the mesh is made of metal.

3. The stent of claim 1, wherein the opening is formed by a discontinuation in the mesh body.

4. The stent of claim 1, wherein the opening is formed by a cutaway section of the mesh body.

5. The stent of claim 1, wherein the opening consists of a number of openings defined by one or more enlarged mesh openings which are larger than the mesh openings of the rest of the mesh.

6. The stent of claim 1, the opening is in a non-disease area of the mesh.

7. The stent of claim 6, wherein the non-disease areas are an end region or both end regions, a central annular region and/or a side region or combination of these, and locates, in use, in a non-disease area of a vessels or bifurcation or in an opening of a bifurcation where two vessels meet.

8. The stent of claim 1, further comprising a marker.

9. The stent of claim 8, wherein the marker is located on a free end of a strand which extends from the metallic mesh.

10. The stent of claim 9, wherein the marker is located inside the opening or at the side of the opening to, in use, indicate to a surgeon the position of the opening.

11. A stent set comprising a stent having a tubular stent body made of the metallic mesh with an opening in the body and a second stent having a tubular stent body made of a mesh; an end of the second stent being receivable into an end of the stent.

12. A sent set as claimed in claim 11 in which The stent set of claim 11, wherein the sent includes a third stent having a tubular stent body made of a mesh.

13. The stent set of claim 11, wherein any one of the opposing ends of the second or third stents are receivable in any one of the open ends of the tubular stent or in the opening in the body of the stent.
